(19) United States
(12) Patent Application Publication
(10) Pub. No.: US 2004/0236270 A1
(43) Pub. Date: Nov. 25, 2004

(54) Device for administering a treatment fluid and the use of such a device for fighting cancer locally in an organ

(76) Inventor: Kokbing Lo, Oldenzaal (NL)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/481,296

(22) PCT Filed: Jun. 21, 2002
(86) PCT No.: PCT/NL02/00414

(30) Foreign Application Priority Data
Jun. 21, 2001 (NL) 1018354

(51) Int. Cl. A61M 1/00; A61M 29/00

(52) U.S. Cl. 604/27; 604/96.01

(57) ABSTRACT

The invention relates to a device for administering a treatment fluid, in particular a chemotherapeutic agent, to an organ comprising at least one inlet vein and one outlet vein, which device comprises: first closing means for closing the at least one inlet vein; second closing means for closing the at least one outlet vein; feed means for feeding the treatment fluid to the organ; drain means for draining the treatment fluid from the organ; a reservoir for storing the treatment fluid; and pumping means for pumping the treatment fluid from the reservoir via the feed means into the organ and out of the organ again via the drain means.

The invention relates to a device for administering a treatment fluid, in particular a chemotherapeutic agent, to an organ comprising at least one inlet vein and one outlet vein.

When a disease, for instance cancer, is present locally in the organ, it is then usual to inject a treatment fluid into body in order to thus combat this local disease. The drawback of injecting the treatment fluid is however that it spreads over the whole body and therefore only part of it arrives at the site where it is required. Particularly in combatting cancer by means of a chemotherapeutic agent, this has major consequences for the patient. The chemotherapeutic agent has an effect on the whole body such that symptoms such as nausea, hair loss and the like occur. Such a treatment can moreover only be applied when the body is in sufficiently healthy state.

Such a method furthermore has the drawback that the maximum possible concentration of the treatment fluid can usually not be achieved in the diseased organ, since other organs of the body would otherwise suffer too much damage from the treatment fluid. Such a method has the further drawback that a relatively large amount of treatment fluid must be used to control the local disease. Since treatment fluids such as a chemotherapeutic agent are expensive, this is disadvantageous.

It is an object of the invention to reduce or even obviate the above stated drawbacks.

This object is achieved with a device for administering a treatment fluid, in particular a chemotherapeutic agent, to an organ comprising at least one inlet vein and one outlet vein, which device comprises:

first closing means for closing the at least one inlet vein;

second closing means for closing the at least one outlet vein;

feed means for feeding the treatment fluid to the organ;

drain means for draining the treatment fluid from the organ;

a reservoir for storing the treatment-fluid; and

pumping means for pumping the treatment fluid from the reservoir via the feed means into the organ and out of the organ again via the drain means.

An organ can temporarily go without oxygen, i.e. can closed off from the blood circulation. This is achieved by means of the closing means. By means of the feed and drain means it thus becomes possible to allow the treatment fluid to flow through the organ, in order to thus treat only the organ. It is thus possible to carry the maximum possible concentration of the treatment fluid into the diseased organ. The organ is as it were flushed through with the treatment fluid. After the maximum closing period for the inlet vein and outlet vein has elapsed, they are opened again, so that blood can once again flow through the organ.

In a preferred embodiment according to the invention at least one of the first and second closing means comprises an inflatable balloon. An inlet vein or an outlet vein can hereby be closed in simple manner.

In another embodiment according to the invention at least one of the feed means and drain means comprises at least one cannula. The treatment fluid can either be fed to or drained from the organ through this cannula.

In yet another embodiment according to the invention at least one cannula extends through the balloon. If the organ has an easily accessible inlet vein or outlet vein, the inflatable balloon can be arranged therein to thus close the outlet vein or inlet vein, and because the cannula extends through this balloon the treatment fluid can be supplied or drained in simple manner on the side of the organ.

If the inlet vein or outlet vein of the organ is a small lateral branch of a large vein, the balloon can then be arranged in the large vein downstream of the branch, while the cannula debouches before it reaches the balloon so that the treatment fluid can be supplied and drained in this manner.

Another preferred embodiment of the device according to the invention further comprises at least one monitoring cannula for monitoring the closure of at least one of the first and second closing means. By a suitable placing of the monitoring cannula it is possible by means of a suction to monitor whether the treatment fluid is seeping along the closing means, such as a balloon. It is thus possible to monitor whether the closure is sufficient.

Yet another preferred embodiment of the device according to the invention further comprises control means for intermittently closing the inlet vein and the outlet vein and administering the treatment fluid. Since an organ can only be closed off from the blood circulation for a limited period of time, it is essential to intermittently close the veins and leave them clear again. The flushing through of the organ with the treatment fluids can be repeated a number of times at a pre-entered interval by means of the control means.

The invention further relates to the use of a device according to the invention for combatting cancer locally in an organ.

These and other features of the invention are further elucidated with reference to the annexed drawings.

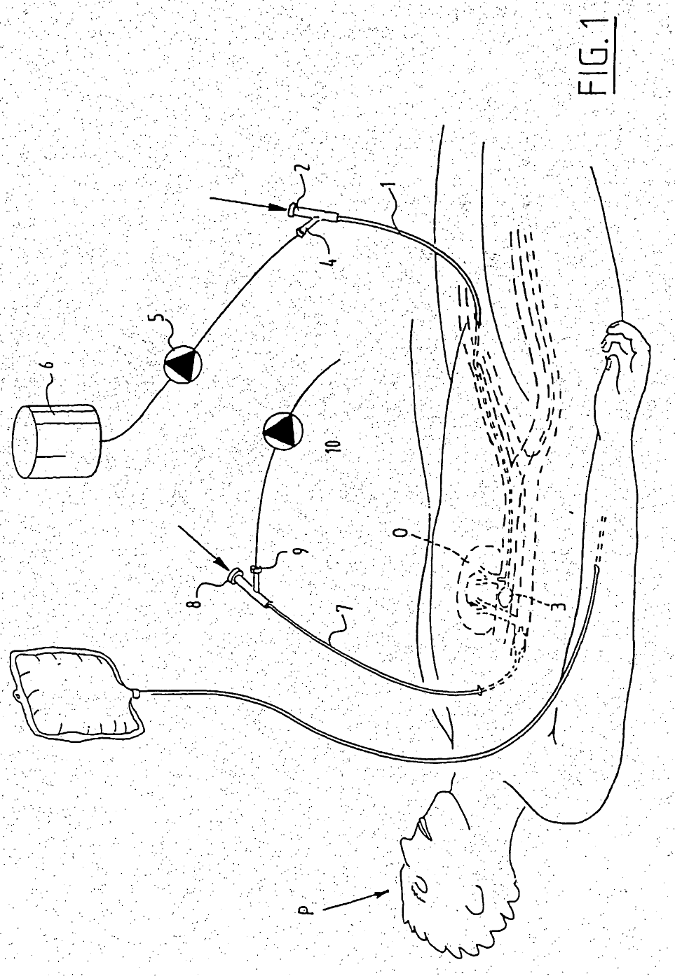

FIG. 1 shows schematically a first embodiment according to the invention.

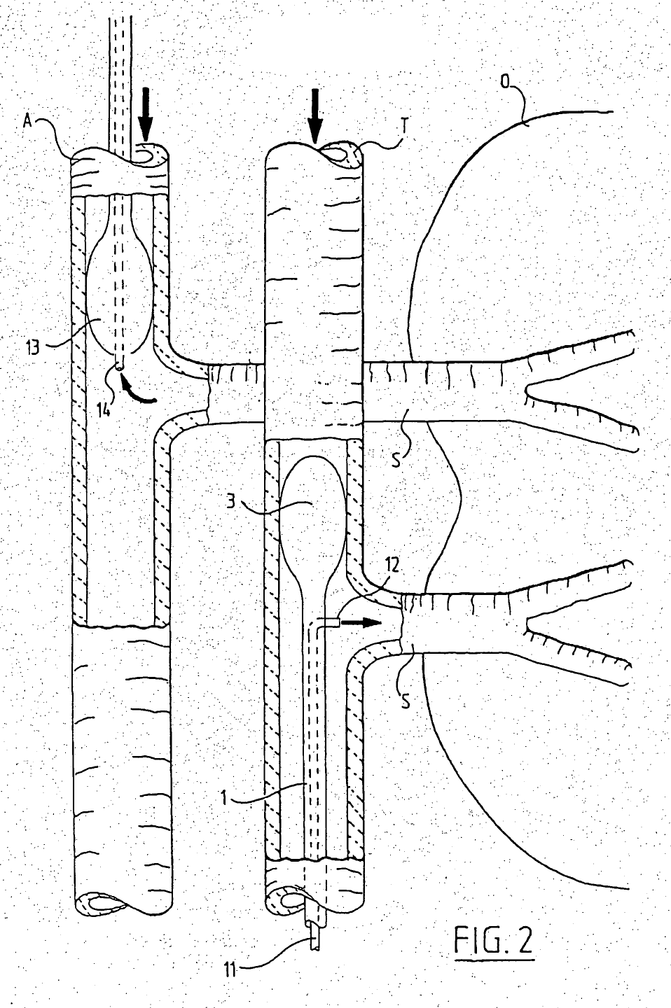

FIG. 2 shows in more detail the closing means of the invention according to FIG. 1.

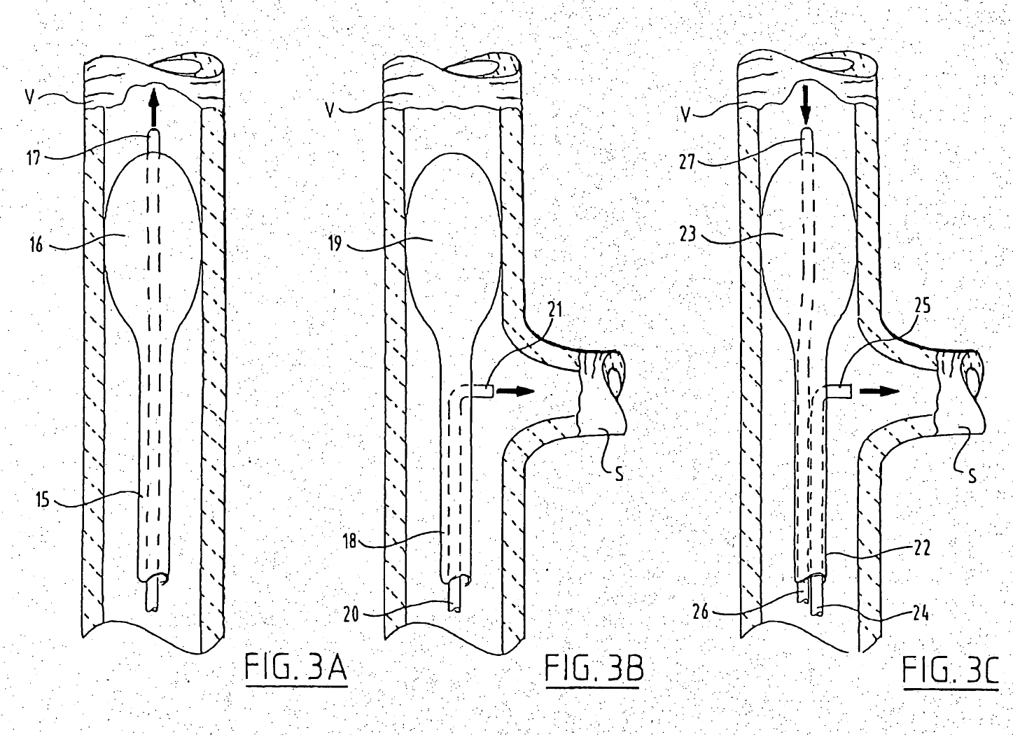

FIGS. 3a-3c show different embodiments of the closing means according to the invention.

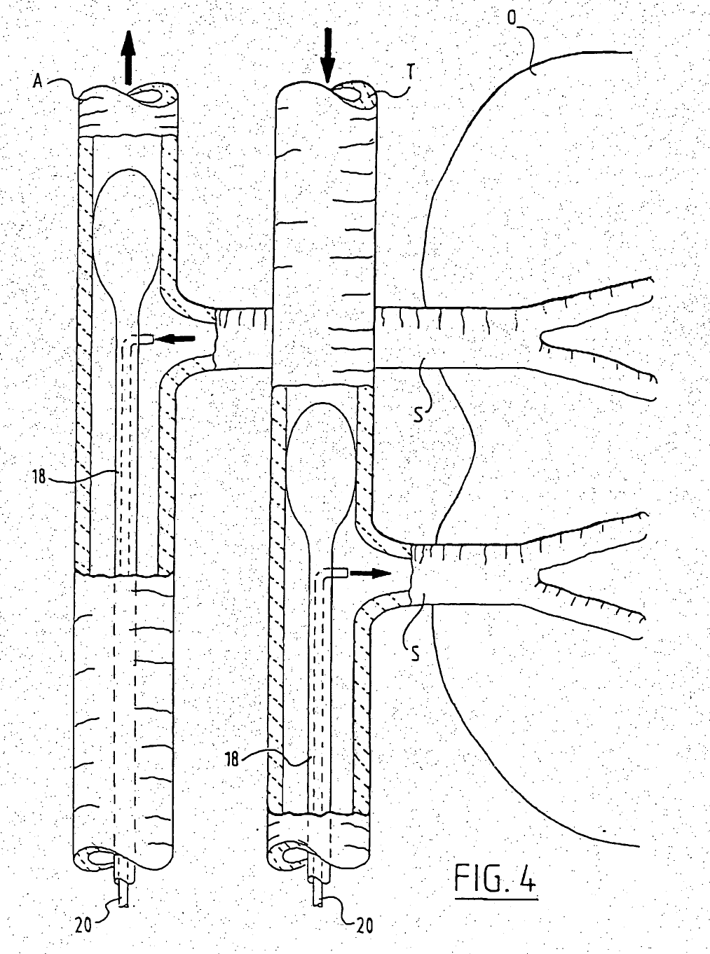

FIG. 4 shows a part of a second embodiment according to the invention.

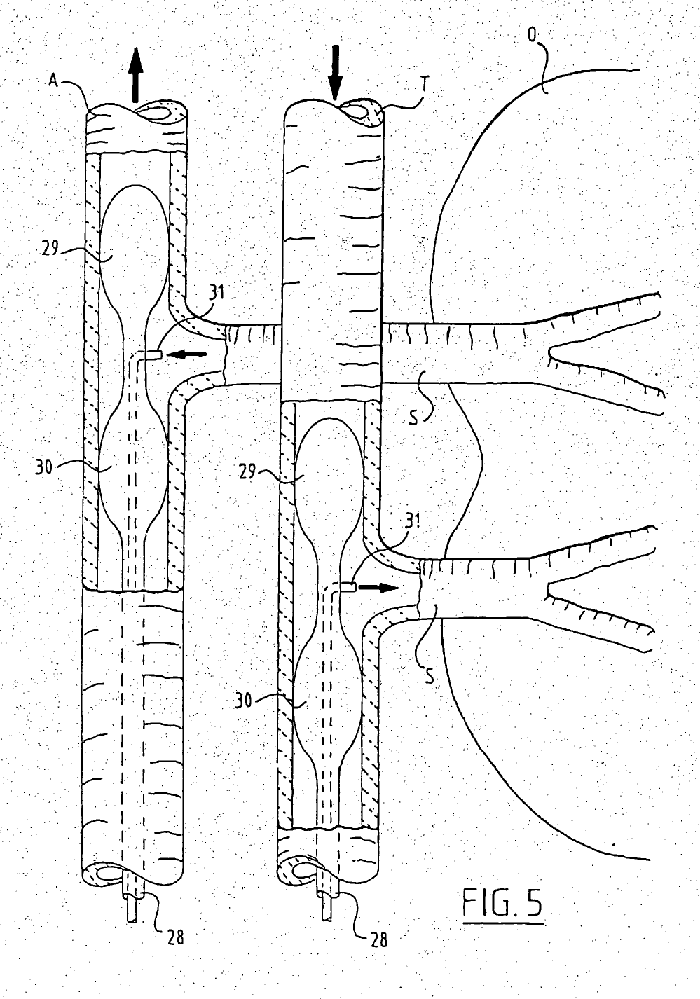

FIG. 5 shows a third embodiment of a device according to the invention.

FIG. 1 shows schematically a patient P, wherein an organ O is present which has to be treated with a fluid. As will be further elucidated below with reference to FIG. 2, a catheter 1 is arranged in an inlet vein, whereby a balloon 3 can be inflated by means of blowing in air via opening 2 in order to thus close the vein. Via the second opening 4 of catheter 1 a treatment fluid can then be pumped from a reservoir 6 to the organ O by means of a pump 5.

A second catheter 7 is arranged in an outlet vein of organ O. Here also a balloon can be inflated by means of blowing air into opening 8 in order to thus close the outlet vein. Via second opening 9 the treatment fluid which has flowed through organ O can be drawn off by means of a pump 10.

FIG. 2 shows in more detail the closing of the veins of organ O. Catheter 1, to which the balloon 3 is fixed, is arranged in inlet vein T. As seen in the direction of flow, this balloon 3 is placed before the branching with organ O. Via the cannula 11 the treatment fluid can thus be introduced into the organ via opening 12.

The catheter 7 is arranged in outlet vein A. This catheter 7 also has a balloon 13 with which the outlet vein is closed. Via cannula 14 treatment fluid can thus be drawn off from organ O, whereby the treatment fluid cannot enter the rest of the body.

FIG. 3a shows an embodiment of the closing means and the feed or drain means. Placed once again in vein V is a catheter 15 on which a balloon 16 is arranged. Through this balloon 16 protrudes a cannula 17 with which treatment fluid can be supplied or drained. This embodiment is advantageous in the case that vein V leads directly to the organ and there are no branches.

In FIG. 3b is shown a second embodiment of the closing means and feed or drain means. A catheter 18 comprising a balloon 19 on its outer end is herein placed in a vein V with a branch S. Further arranged in catheter 18 is a cannula 20 which debouches by means of opening 21 into branch S before it reaches balloon 19.

Finally, FIG. 3c shows a third embodiment of a catheter 22. This catheter 22 once again has a balloon 23 for closing vein V and a first cannula 24 debouching into branch S by means of openings 25. A second cannula 26 is further arranged in catheter 22 and this cannula 26 extends through balloon 23, so that fluid can be drawn off via opening 27 in order to monitor whether any treatment fluid is flowing out of opening 25 and along balloon 23.

FIG. 4 shows the use of two catheters 18 according to FIG. 3b. Inlet vein T and outlet vein A are hereby closed and the treatment fluid can be supplied and drained by means of cannulas 20.

An alternative embodiment of two catheters 28 is shown in FIG. 5. These two catheters 28 are arranged in inlet vein T and outlet vein A. Each catheter 28 has two balloons 29 and 30 with which the relevant vein can be closed. A cannula 31 debouches between the two balloons 29 and 30. Cannula 31 can debouch at the position of branch S by suitable placing of balloons 29 and 30. Using such a catheter 28 the branch can be insulated and it is possible to ensure that the treatment fluid is supplied to and drained again from only an organ O.

Such a device can of course also be used for locally administering for instance a contrast fluid.

If the maximum period of time for which an organ can go without oxygen is smaller than the time required for flushing-through, it is then essential to first of all determine the flushing time of the organ. This can be done by adding a so-called marker. This is a substance which can be easily traced. As soon as the marker is detected on the draining side, the flushing time of the organ is firmly established.

As described above, the inlet vein can now be closed and the treatment fluid guided through the organ. Only after the flushing time has elapsed will the outlet vein be closed in order to collect the treatment fluid. When the maximum closing time has been reached the inlet vein is opened again and oxygen supplied to the organ via the blood. The outlet vein is then opened after a period equal to the flushing time, since only then does the blood flow out of the organ again. This is then repeated a number of times.

1-7. (cancelled)

8. A device for administering a treatment fluid, such as a chemotherapeutic agent, to an organ, said device comprising:
(a) at least one inlet vein and one outlet vein, which device comprises:
(b) a first closing means for closing the at least one inlet vein;
(c) a second closing means for closing the at least one outlet vein;
(d) feed means for feeding the treatment fluid to the organ;
(e) drain means for draining the treatment fluid from the organ;
(f) a reservoir for storing the treatment fluid; and
(g) pumping means for pumping the treatment fluid from the reservoir via the feed means into the organ and out of the organ again via the drain means, and
(h) control means for intermittently closing the inlet vein and outlet vein and administering the treatment fluid.

9. The device as claimed in claim 8, wherein at least one of the first and second closing means comprises an inflatable balloon.

10. The device as claimed in claim 8, wherein at least one of the feed means and drain means comprises at least one cannula.

11. The device as claimed in claim 9, wherein at least one of the feed means and drain means comprises at least one cannula.

12. The device as claimed in claim 11, wherein the at least one cannula extends through the balloon.

13. The device as claimed in claim 11, further comprising at least one monitoring cannula for monitoring the closure of at least one of the first and second closing means.
